(19) United States
(12) Patent Application Publication
(10) Pub. No.: US 2004/0054350 A1
(43) Pub. Date: Mar. 18, 2004

(54) Enteral feeding unit having a reflux device and reflux method

(76) Inventors: Michael C. Shaughnessy, Arlington Heights, IL (US); Jason L. Wellet, Chicago, IL (US)

Correspondence Address: BELL, BOYD & LLOYD, LLC, PO BOX 1135, CHICAGO, IL, 60690-1135, US

(21) Appl. No.: 10/246,436

(22) Filed: Sep. 17, 2002

(51) Int. Cl. A61M 25/16

(52) U.S. Cl. 604/535; 604/500

(57) ABSTRACT

The present invention relates to an enteral feeding unit having a reflux device for use during enteral feeding. The enteral feeding unit, in one embodiment, includes an administration device connected to a relatively large reservoir tube which is in fluid communication with a patient's oral or nasal passage. The reservoir tube receives the reflux fluid from a patient and returns the reflux fluid to the patient when certain pressure conditions are present. This reservoir tube is preferably non-collapsible and preferably bears volumetric markings which enable a user to measure ,the amount of reflux fluid expelled by a patient. This type of enteral feeding unit provides users with a convenient way of accommodating and monitoring reflux fluid during the enteral feeding process.

### CROSS REFERENCE TO RELATED APPLICATIONS

This application relates to the following co-pending commonly owned patent application: U.S. patent application Ser. No. 09/665,194, filed on Sep. 18, 2000, entitled “Apparatus and Method for Relieving Gastric Pressure During Enteral Feeding.”

### BACKGROUND OF THE INVENTION

Enteral nutrition is a form of hyperalimentation and metabolic support in which nutrient formulas or medicaments are delivered directly to the gastrointestinal tract, either the stomach or the duodenum. Nutrient administration is accomplished through use of an enteral feeding system or device. Certain enteral feeding devices include pumps which deliver feeding fluid to the patient. Other enteral feeding devices rely upon gravity to move the feeding fluid from a container (suspended above patient level) to the patient.

During enteral feeding, excessive gastric pressure may result from the accumulation of gas or liquid resulting from stomach contractions, movement of the patient's abdomen, crying or through normal formation of gas. From time to time, the body relieves such excess gastric pressure by expelling gas or liquid or reflux fluid. The term, “reflux fluid” as used herein includes any gas, any liquid, any partially solid and liquid substance or any material which the body can expel.

Typically the expulsion of reflux fluid occurs during a burping response in which reflux fluid is expelled upward from the stomach through the esophagus and is expressed out of the mouth, where the enteral feeding tube is orally intubated or through the nasal passages, where naso-pharyngeal intubation has been utilized.

When the patient expels reflux fluid, the reflux fluid often flows out of the patient's mouth or the nose because the enteral feeding device is not adapted to receive the back flow of reflux fluid. Specifically, the feeding fluid pressure in the enteral feeding device prevents reflux fluid from flowing from the patient into the patient feeding tube. Though gastric reflux pressure created by even limited episodes of stomach movement or crying may exceed several feet of water, such reflux pressure can be inadequate to overcome the greater forward fluid pressure present within the patient feeding tube. As a result, expelled reflux fluid can accumulate in or around the nasal or oral passages.

This accumulation of reflux fluid is undesirable because the patient loses feeding fluid, and moreover, it is possible for the patient to inhale the reflux fluid into the lungs with possible risk of aspiration pneumonia or other ailments. The problem of gastric reflux pressure and reflux fluid is most acute in neonates, infants and small children in which gastric pressure may rapidly accumulate through periodic episodes of crying and because such patients have yet to develop control over the burping response as a means of gastric pressure relief. However, it is not unusual for adult patients undergoing enteral feeding to experience occasional difficulties with gastric reflux pressure relief.

There is a known gastric pressure relief device which is used in conjunction with enteral feeding devices. Such relief device includes a bag connected to a tube which is in fluid communication with the feeding device tube and the patient feeding tube. The bag vents to the atmosphere. If a patient expels enough reflux fluid, the bag eventually fills with reflux fluid, and any gas can escape to the atmosphere. Depending upon the pressure conditions during feeding, the reflux fluid can remain in the bag or flow back to the patient for feeding. One disadvantage with such relief device is the difficulty or inability for users to measure the amount of reflux fluid. Another disadvantage with such relief device is the difficulty in tracking changes of volume in the bag due to the irregular shape and the size of the bag. This adversely impacts accurate enteral administration of fluid nutrient formula, particularly since a selected quantity of nutrient formula is administered over a given period of time. Therefore, there is a need to overcome such disadvantages.

### SUMMARY OF THE INVENTION

The enteral feeding unit, in one embodiment of the present invention, includes an administration device, such as a feeding bag and pump set, connected to a reflux device which, in turn, is connected to a feeding tube received by the patient. In one embodiment, the reflux device includes a relatively large, cylindrical reservoir tube, preferably non-collapsible, which collects and returns reflux fluid to patients. The reservoir tube bears a plurality of volumetric marks which enable users to track the amount of reflux fluid which patients expel over time. Since the reflux device is comprised completely of tubes and tube connectors, the reflux device is relatively simple to manufacture, assemble and use. Specifically, the cylindrical shaped reservoir tube facilitates a user's reading and tracking of changes in the volume of reflux fluid.

In one embodiment, the reflux device of the present invention includes a reservoir tube having a first end defining at least one opening, a body having a predetermined diameter and a second end defining an opening. The delivery tube includes a first end connected to the second end of the reservoir tube, a branch adapted for connection to an administration tube and a second end received directly or indirectly by a patient. The delivery tube has a diameter which is less than the diameter of the body of the reservoir tube. The reservoir tube is preferably non-collapsible and large enough to receive all of the reflux fluid expelled by a patient during an entire enteral feeding process.

In operation of one embodiment, a user enterally feeds a patient by delivering feeding fluid from a feeding source through a delivery tube to a patient, receiving reflux fluid from the patient through the delivery tube, channeling the received reflux fluid to a reservoir tube and enabling any gaseous portion of the reflux fluid to vent to the atmosphere.

It is therefore an advantage of the present invention to provide an enteral feeding unit having a reflux device and reflux method.

Another advantage of the present invention is to provide a non-collapsible, cylindrical reservoir for reflux fluid expelled during enteral feeding.

Yet another advantage of the present invention is to provide a volumetrically marked reservoir for reflux fluid which is relatively simple and convenient to read during enteral feeding.

Still another advantage of the present invention is to provide a reflux device which is relatively simple to construct and manufacture.

Yet another advantage of the present invention is to provide a reflux device which provides a relatively high level of convenience and accuracy in gauging volume and volume changes in reflux fluid during enteral feeding.

Another advantage of the present invention is to enable users to provide enhanced health care during enteral feeding in the areas of gastric pressure relief, the measurement of reflux fluid and the recovery and return of reflux fluid to patients, such as neonatal patients.

Additional features and advantages of the present invention are described in, and will be apparent from, the following Detailed Description of the Invention and the figures.

### BRIEF DESCRIPTION OF THE FIGURES

FIG. 1 is a side elevation view of the enteral feeding unit in one embodiment of the present invention.

FIG. 2 is a fragmentary side elevation view of a multi-way connector of the reflux device of the enteral feeding unit in one alternative embodiment of the present invention.

FIG. 3 is a side elevation view of the enteral feeding unit in one alternative embodiment of this present invention.

FIG. 4 is a fragmentary side elevation view of a multi-way connector of the reflux device of the enteral feeding unit in one alternative embodiment of the present invention.

### DETAILED DESCRIPTION OF THE INVENTION

Referring now to the drawings, FIG. 1 illustrates one embodiment of enteral feeding unit 10 which includes an administration device 12 removably connected to a reflux device 14. The administration device 12 is used for administering feeding fluid to a patient, and the reflux device 14 receives, retains and returns reflux fluid to the patient. The administration device 12 can include any suitable device or mechanism which distributes or delivers feeding fluid to the patient. In one embodiment, the administration device 12 includes: (a) a feeding bag 16 connected to administration tubing 18 having administration tubes 18a and 18b; (b) a pump 20 which produces a suction force within administration tubing 18 (preferably by regularly stretching and releasing a portion of administration tubing 18); and (c) a slideable tube set clamp 22 connected to the administration tube 18a utilized to cause the administration device 12 to become inoperative.

The pole 24 supports the feeding bag 16 and the pump 20. In operation, the feeding bag 16 contains a supply of feeding fluid, and the pump 20 draws the feeding fluid from feeding bag 16 and forces the feeding fluid through the administration tubing 18. It should be appreciated that enteral feeding unit 10 can include any suitable type of administration feeding device or pump device other than the administration feeding device 12. For example, alternate administration feeding devices may not include a pump but instead such devices may rely upon gravity to distribute feeding fluid to the patient.

The reflux device 14, removably connected to the administration device 12, includes: (a) a reservoir tube 26 which receives reflux fluid from the patient, retains the reflux fluid and returns the reflux fluid to the patient; and (b) a delivery tube 28 connected to the reservoir tube 26 which brings the reservoir tube 26 into fluid communication with the administration tubing 18 and the feeding tube 30.

The reservoir tube 26 includes: (a) a cover or vent member 32 connected to the upper end of reservoir tube 26 for controlling the entrance of debris and other material into the reservoir tube 26 and for enabling reflux gas to escape to the atmosphere; (b) a hook or other engaging member 34 attached to the upper end of the reservoir tube 26 for hanging the reservoir tube 26 from the pole 24; (c) a plurality of markings 36 along the reservoir tube 26; and (d) a funnel-shaped portion or adapter member 38 which enables the reservoir tube 26 to be rigidly connected to the delivery tube 28.

The reservoir tube 26 is preferably non-collapsible, cylindrical in shape, transparent or translucent, and constructed of a suitable flexible plastic, such as polyethylene. The diameter of reservoir tube 26 is preferably in the range of five to ten times the diameter of the feeding tube 30. In one embodiment, the reservoir tube 26 is sixty-four inches in length and has a diameter size of thirty-three Fr. on the French scale and a volume of one hundred forty cubic centimeters.

The cover or vent member 32 includes a surface which overlays the upper, open end of the reservoir tube 26. The cover or vent member 32 preferably is a suitable filter which prevents dust and debris from entering the reservoir tube while enabling gastric and reflux gases to escape to the atmosphere. It should be appreciated that in other embodiments, the cover member can include a vent, a valve or any porous material. It should also be appreciated that the present invention need not include a cover member. For example, in one embodiment, the reservoir tube's upper end can be downwardly curved so that the opening of the upper end is pointed downward. This curved construction minimizes the entrance of dust or debris into the reservoir tube 26 while enabling gastric gases to freely escape to the atmosphere.

In another embodiment, the upper end of the reservoir tube 26 can be fully enclosed with a stopper or cap, and one or more holes or orifices can be constructed within the wall of the upper portion of the reservoir tube 26. Such a construction minimizes the entrance of dust or debris into the reservoir tube 26 while enabling gastric gases to freely escape to the atmosphere.

The hook or engaging member 34, which enables the reservoir tube 26 to connect to and hang from the device pole 24, is rigidly connected to the outer wall of the upper portion of the reservoir tube 26. Preferably, the engaging member 34 is a hook member constructed of a suitable rigid-forming plastic, and the engaging member 34 is connected to the reservoir tube 26 through a molding or adhesive process. The engaging member 34 can, however, be constructed of any suitable size, shape and material, and the engaging member 34 can be fastened to reservoir tube 26 in any suitable fashion.

The delivery tube 28, connected to the lower end of the reservoir tube 26, includes a multi-way connector, three-way connector or Y-port connector 40 positioned between an upper delivery tube portion 42 and a lower delivery tube portion 44. The delivery tube 28 also includes a step connector 39 at the proximal end of the lower delivery tube portion 44 for removably connecting the delivery tube 28 to the feeding tube 30. Preferably, the upper delivery tube portion 42, Y-port connector 40 and lower delivery tube portion 44 are permanently connected to one another through a suitable adhesive or molding technique.

The Y-port connector 40 includes an administration branch 41 which receives administration tube 18b. The Y-port connector 40 also has flexible arms 46 and 48. Flexible arm 46 has a cap or stopper 47 for sealing and unsealing administration branch 41. Flexible arm 48 has a converter or adapter member 49 which enables administration tubes 18b of different sizes to be connected to administration branch 41.

The upper delivery tube portion 42 of the delivery tube 28 functions as an extension which facilitates the connection of the reservoir tube 26 to the feeding tube 30. It should be appreciated, however, that the present invention need not include the upper delivery tube portion 42 of the reflux tube 28. In such case, the reservoir tube 26 can be connected directly to the Y-port connector 40.

The feeding tube 30 includes a three-way connector or Y-port connector 52 connected to the distal end 30a of the feeding tube 30, and the proximal end 30b of the feeding tube 30 is received by the nasal or oral passages of a patient 54. The Y-port connector 52 includes a multi-purpose branch 52a which enables users to flush out the feeding tube 30, administer medication to patients or conduct other enteral feeding-related activities. Y-port connector 52 also includes two flexible arms 56 and 58 having stoppers 56a and 58a, respectively. Stopper 56a can be used to seal and unseal the multi-purpose branch 52a, and stopper 58a can be used to seal and unseal the distal end 30a of the feeding tube 30.

It should be appreciated that in one alternative embodiment of the present invention, the reflux device does not need to include the Y-port connectors or the feeding tube. Instead, the reflux device can include the reservoir tube which is suitably in fluid communication with one delivery tube or a series of connected delivery tubes. The distal end of the delivery tube or delivery tube series is connected to the reservoir tube, and the proximal end of the delivery tube or delivery tube series is received by the patient. Such delivery tube or delivery tube series is in suitable fluid communication with the administration tube 18b. The term “delivery tube,” as used herein, includes any tube which can direct the flow of fluid.

It should also be appreciated that in the embodiment illustrated in FIG. 1, the reflux device 14 can be used with different types of administration devices 12 with different sized administration tubes 18b. To change administration devices 12, the user can remove the administration tube 18b from the administration branch 41. The user can then connect the administration tube of the new administration device to the administration branch 41. Depending upon the diameter of the new administration tube, the user may or may not need to use adapter member 49 to make the connection to the administration branch 41.

Accordingly, in this embodiment, the administration device and reflux device can be distributed and commercialized separately or as a combination.

In addition, it should be understood that the feeding tube 30 is merely one example of the type of feeding tube which can be used in conjunction with the reflux device of the present invention. The reflux device of the present invention can be used in conjunction with any suitable feeding tube or patient delivery tube.

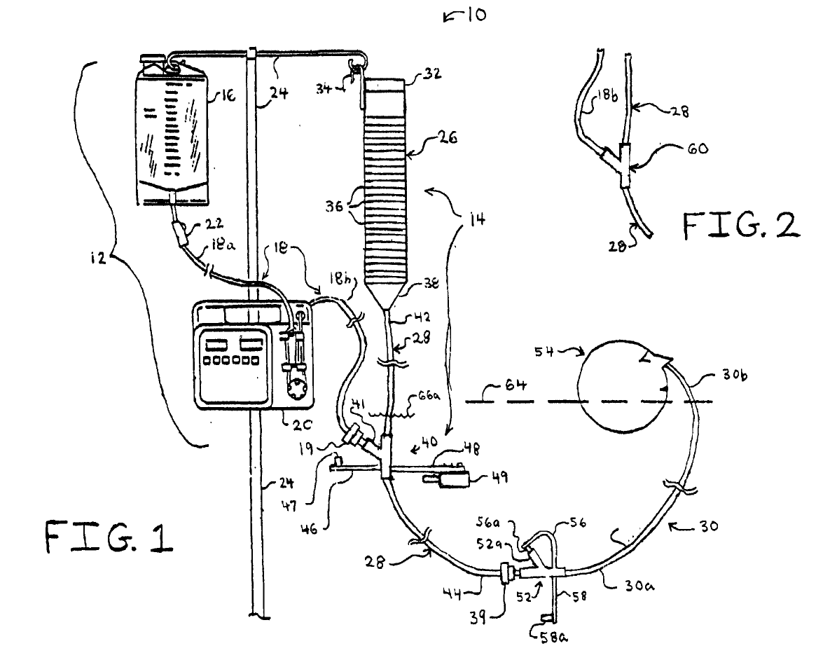

In one embodiment, illustrated in FIG. 2, the administration device 12 and the reflux device 14 are permanently connected to each other. Instead of Y-port connector 40, 25 the reflux device 14 includes the Y-port connector 60. The Y-port connector 60 is permanently connected to the administration tube 18b, preferably through a suitable adhesive, molding process or other suitable fastening technique. Accordingly, in this embodiment, the administration device and reflux device can be distributed and commercialized together as a combination or kit.

Referring back to FIG. 1, the reservoir tube 26 includes a plurality of volumetric markings 36 which enable the user to measure the volume of reflux fluid expelled by a patient. These markings 36 also enable the user to measure changes in the amount of reflux fluid flowing to and from the patient. With this measurement information, users can determine the amount of feeding fluid a patient has ingested without expulsion. Because the reservoir tube 26 may carry a meaningful quantity of formula, particularly, for neonatal patients, the markings 36 enable the user to monitor the amount of refluxed formula to ensure that the neonatal patient is receiving the prescribed amount of formula. It should also be appreciated that the present invention can include markings for fluid measurement purposes on any of the delivery tubes, including, but not limited to, the feeding tube.

The markings 36 can include or incorporate any suitable marking or marking system. In one embodiment, markings 36 are lines printed or painted on the reservoir tube 26. In another embodiment, markings 36 are suitable decals affixed to the reservoir tube 26 or suitable impressions, engravings, grooves or other shape modifications to the reservoir tube 26 which a user can use to measure the volume or volume change of reflux fluid.

To install and use the enteral feeding unit 10 of the present invention, the user suspends the feeding bag 16 from the pole 24 and feeds the administration tubing 18 into the pump 20. The user then suspends the reservoir tube 26 from the pole 24. The user inserts the step connector 19 of the administration tube 18a into the administration branch 41 of the Y-port connector 40. Next, the user inserts step connector 39 into the Y-port connector 52 of the feeding tube 30. The user then seals off the multi-purpose branch 52a of the Y-port connector 52 using the stopper 56a of the arm 56. The user then primes the feeding tube 30 by delivering a relatively small amount of feeding fluid to ensure there are no occlusions blocking the flow of feeding fluid to the patient. After any occlusions have been overcome, the user administers feeding fluid to the patient using the pump 20.

The reflux device 14 is essentially an open system because the reservoir tube 26 is vented to ambient atmospheric pressure. Therefore, to prevent air from entering into the feeding tube 30 and reaching the patient, the reflux device 14 is preferably oriented so that there is a constant presence of a relatively small column of feeding fluid (i.e., nutrient formula) located above the Y-port connector 40.

One way in which this can be accomplished is by positioning the Y-port connector 40 at or slightly below the level of the patient's diaphragm or stomach. This patient level is illustrated in FIG. 1 by broken line 64. By positioning the Y-port connector 40 in this manner, the distal end of the delivery tube 28 is preferably located at or slightly below the patient level. With the delivery tube 28 having this position, a small column of feeding fluid remains suspended within a distal portion of delivery tube 28 with the meniscus of the column below the patient level, as illustrated by wavy line 66a in FIG. 1. This column of fluid prevents air from being drawn into the enteral feeding tube 30 and ultimately reaching the patient.

Another unique aspect of maintaining the fluid column is that the height of such column has been observed to fluctuate in response to greater or lesser gastric pressure (ambient atmospheric pressure being a relative constant). Increases in gastric pressure or partial occlusions of the feeding tube may cause the height of the fluid column to increase. Accordingly, by reading the markings 36 a user can gather valuable information about the performance of the enteral feeding unit 10, the patient's gastric pressure and reflux responses and the overall enteral feeding process for a particular patient.

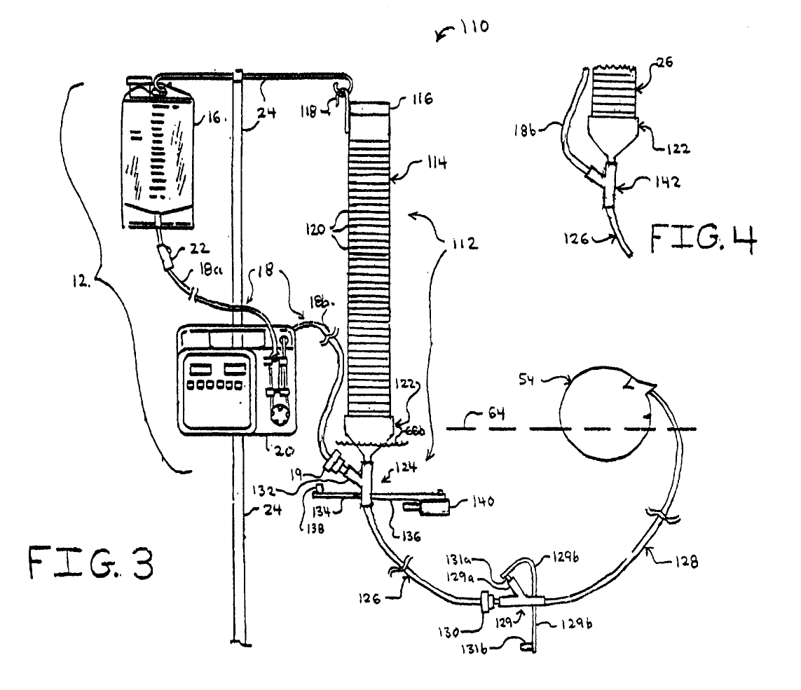

In one alternative embodiment illustrated in FIG. 3, the enteral feeding unit 110 includes the administration device 12 removably connected to the reflux device 112. The reflux device 112 includes a reservoir tube 114 which receives reflux fluid from the patient, retains the reflux fluid and returns the reflux fluid to the patient. The reservoir tube 114 includes: (a) a cover or vent member 116 connected to the upper end of reservoir tube 114 for controlling the entrance of debris and material into the reservoir tube 114 and enabling reflux gas to escape to the atmosphere; (b) a hook or other engaging member 118 attached to the upper end of the reservoir tube 114 for hanging or suspending the reservoir tube 114 from the pole 24; (c) a plurality of markings 120 along the reservoir tube 114; and (d) a funnel-shaped portion or adapter member 122 which enables the reservoir tube 114 to be connected to the Y-port connector 124 of the delivery tube 126. The adapter member 122 can be removably or permanently connected to the body of the reservoir tube 114 using any suitable fastening technique, including, but not limited to, adhesion, molding, step-connection or press-fitting.

The reservoir tube 114 is preferably non-collapsible, cylindrical in shape, transparent or translucent, and constructed of a suitable flexible plastic, such as polyethylene. The diameter of reservoir tube 114 is preferably in the range of five to ten times the diameter of the feeding tube 128. In one embodiment, the reservoir tube 114 has a suitable length and a diameter size of thirty-three Fr. on the French scale and a volume of one hundred forty cubic centimeters.

The cover or vent member 116 includes a surface which overlays the upper, open end of the reservoir tube 114. The cover or vent member 116 preferably is a suitable filter which prevents dust and debris from entering the reservoir tube while enabling gastric and reflux gases to escape to the atmosphere. It should be appreciated that in other embodiments, the cover member can include a vent, a valve or any porous material. It should also be appreciated that the present invention need not include a cover member. For example, in one embodiment, the reservoir tube's upper end can be downwardly curved so that the opening of the upper end is pointed downward. This curved construction minimizes the entrance of dust or debris into the reservoir tube 114 while enabling gastric gases to freely escape to the atmosphere.

In another embodiment, the upper end of the reservoir tube 114 can be fully enclosed with a stopper or cap, and one or more holes or orifices can be constructed within the wall of the upper portion of the reservoir tube 114. Such a construction minimizes the entrance of dust or debris into the reservoir tube 114 while enabling gastric gases to freely escape to the atmosphere.

The engaging member 118, which enables the reservoir tube 114 to connect to and hang from the pole 24, is rigidly connected to the outer wall of the upper portion of the reservoir tube 114. The delivery tube 126, connected to the reservoir tube 114, includes a sleeve, multi-way connector or Y-port connector 124 at the distal end of the delivery tube 126 for fluid communication with the administration tube 18a. The delivery tube 126 has a step connector 130 at the proximal end of the delivery tube 126 for removably connecting the delivery tube 126 to the feeding tube 128. Preferably, the Y-port connector 124 and step connector 130 are permanently connected to the delivery tube 126 through a suitable adhesive or molding technique.

The Y-port connector 124 can be removably or permanently connected to the adapter member 122 of the reservoir tube 114 using any suitable fastening technique, including, but not limited to, adhesion, molding, step-connection or press-fitting. The Y-port connector 124 includes an administration branch 132 which receives administration tube 18b, and the Y-port connector 124 has flexible arms 134 and 136. Flexible arm 136 has a cap or stopper 138 for sealing and unsealing administration branch 132. Flexible arm 134 has a converter or adapter member 140 which enables administration tubes 18a of different sizes to be connected to administration branch 132.

The feeding tube 128 preferably includes a Y-port connector 129 at the distal end of the feeding tube 128, and the proximal end of the feeding tube 128 is received by a patient 54. The Y-port connector 129 includes a multi-purpose branch 129a for flushing and other purposes and two flexible arms 129b having stoppers 131a and 131b. The stopper 131 a can be used to seal and unseal the multi-purpose branch 129a, and the stopper 131b can be used to seal and unseal the distal end of the feeding tube 128. It should be understood that the feeding tube 128 is merely one example of the type of feeding tube which can be used in conjunction with the reflux device of the present invention. The reflux device of the present invention can be used in conjunction with any suitable feeding tube or patient delivery tube.

It should be appreciated that in the embodiment illustrated in FIG. 3, the reflux device 112 can be used with different types of administration devices 12 with different sized administration tubes 18b. To change administration devices 12, the user can remove the administration tube 18b from the administration branch 132. The user can then connect the administration tube of the new administration device to the administration branch 132. Depending upon the diameter of the new administration tube, the user may or may not need to use adapter member 140 to make the connection to the administration branch 132. Accordingly, in this embodiment, the administration device and the reflux device can be distributed and commercialized separately or as a combination.

In one embodiment, illustrated in FIG. 4, the administration device 12 and the reflux device 112 are permanently connected to each other. Instead of Y-port connector 124, the reflux device 112 includes the Y-port connector 142. The Y-port connector 142 is permanently connected to the administration tube 18b, preferably through a suitable adhesive, molding process or other suitable fastening technique. Accordingly, in this embodiment, the administration device and reflux device can be distributed and commercialized together as a combination or kit.

Referring back to FIG. 3, the reservoir tube 114 includes a plurality of volumetric markings 120 which enable the user to measure the volume of reflux fluid expelled by a patient. The markings 120 also enable the user to measure changes in the amount of reflux fluid flowing to and from the patient. With this measurement information, users can determine the amount of feeding fluid a patient has ingested without expulsion. Because the reservoir tube 114 may carry a meaningful quantity of formula, particularly, for neonatal patients, the markings 120 enable the user to monitor the amount of refluxed formula to ensure that the neonatal patient is receiving the prescribed amount of formula. It should also be appreciated that the present invention can include markings for fluid measurement purposes on any of the delivery tubes, including, but not limited to, the feeding tube.

The markings 120 can include or incorporate any suitable marking or marking system. In one embodiment, the markings 120 are lines printed or painted on the reservoir tube 114. In another embodiment, the markings 120 are suitable decals affixed to the reservoir tube 114 or suitable impressions, engravings, grooves or other shape modifications to the reservoir tube 114 which a user can use to measure the volume or volume change of reflux fluid.

To install and use the enteral feeding unit 110 of the present invention, the user suspends the feeding bag 16 from the pole 24 and feeds the administration tubing 18 into the pump 20. The user then suspends the reservoir tube 114 from the pole 24. The user inserts the step connector 19 of the administration tube 18b into the administration branch 132 of the Y-port connector 124. Next, the user inserts the step connector 130 into the Y-port connector 129 of the feeding tube 128. The user then seals off the multi-purpose branch 129a of the Y-port connector 129 using the stopper 131a of an arm 129b. The user then primes the feeding tube 128 by delivering a relatively small amount of feeding fluid to ensure there are no occlusions blocking the flow of feeding fluid to the patient. After any occlusions have been overcome, the user administers feeding fluid to the patient using the pump 20.

The reflux device 112 is essentially an open system because the reservoir tube 114 is vented to ambient atmospheric pressure. Therefore, to prevent air from entering into the feeding tube 128 and reaching the patient, the reflux device 112 is preferably oriented so that there is a constant presence of a relatively small column of feeding fluid (i.e., nutrient formula) located above the Y-port connector 124.

One way in which this can be accomplished is by positioning the Y-port connector 124 at or slightly below the level of the patient's diaphragm or stomach. This patient level is illustrated in FIG. 3 by broken line 64. By positioning Y-port connector 124 in this manner, the distal end of the delivery tube 126 is preferably located at or slightly below the patient level.

With the delivery tube 126 having this position, a small column of feeding fluid remains suspended within a distal portion of delivery tube 126 with the meniscus of the column below the patient level 64, as illustrated by wavy line 66b in FIG. 3. This column of fluid prevents air from being drawn into the enteral feeding tube 128 and ultimately reaching the patient.

Another unique aspect of maintaining the fluid column is that the height of such column has been observed to fluctuate in response to greater or lesser gastric pressure (ambient atmospheric pressure being a relative constant). Increases in gastric pressure or partial occlusions of enteral feeding tube may cause the height of the fluid column to increase. Accordingly, by reading the markings 120 a user can gather valuable information about the performance of the enteral feeding unit 110, the patient's gastric pressure and reflux responses and the overall enteral feeding process for a particular patient.

In the course of normal operation of the enteral feeding unit of the present invention, the height of the column of fluid within the reservoir tube may fluctuate when the enteral feeding tube becomes occluded during feeding or when the patient accumulates excessive gastric pressure, fluid, gas or other reflux fluid. In the latter case, the reflux fluid flows from the patient's stomach through the feeding tube into the reservoir tube. Any gas within the reflux fluid will separate and escape through the cover member of the reservoir tube.

The enteral feeding unit of the present invention, in one embodiment, includes a relatively large, cylindrical, non-collapsible reservoir tube which bears volumetric markings. The reservoir tube collects reflux fluid expelled by a patient, and the reservoir tube returns the reflux fluid to the patient when predetermined pressure conditions are satisfied. This reservoir tube, which is used in conjunction with an administration device, facilitates the accurate measurement and assessment of the amount of feeding fluid ingested by a patient (without expulsion), the amount of reflux fluid expelled by a patient and the change in the amount of reflux fluid expelled or recovered by a patient over time. The enteral feeding unit of the present invention enables users to enhance the effectiveness of enteral feeding, accommodate reflux fluid, minimize ailments and health risks caused by reflux fluid and accurately and conveniently measure the quantity of reflux fluid expelled by a patient.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

The invention is claimed as follows:

1. A reflux device for use during enteral feeding, comprising:
a reservoir tube having a first end defining at least one opening, a body having a predetermined diameter and a second end defining an opening; and
a delivery tube including a first end connected to the second end of the reservoir tube, a branch adapted for connection to an administration tube and a second end received directly or indirectly by a patient, the delivery tube having a diameter less than the diameter of the body of the reservoir tube.

2. The reflux device of claim 1, wherein the reservoir tube is non-collapsible.

3. The reflux device of claim 1, wherein the reservoir tube has a volume which is large enough to receive all reflux fluid expelled by a patient during an entire enteral feeding process.

4. The reflux device of claim 1, which includes a plurality of measurement markings on the reservoir tube.

5. The reflux device of claim 1, which includes a vent member connected to the first end of the reservoir tube.

6. The reflux device of claim 1, which includes a feeding tube connected to the delivery tube, wherein the feeding tube is received by the patient.

7. The reflux device of claim 1, wherein the branch has a position at or below a patient level.

8. A reflux device for use with an administration device during enteral feeding of a patient, the administration device having at least one administration tube, the reflux device comprising:
a non-collapsible reservoir tube having a first end defining at least one opening which vents to atmosphere, a body having a predetermined diameter and a second end defining an opening having a diameter less than the diameter of the body;
a plurality of measurement markings on the non-collapsible reservoir tube; and
a delivery tube, in fluid communication with the reservoir tube and the administration tube, which is received directly or indirectly by the patient, the delivery tube having a diameter less than the diameter of the body of the reservoir tube.

9. The reflux device of claim 8, wherein the reservoir tube has a volume which is large enough to receive all reflux fluid expelled by a patient during an entire enteral feeding process.

10. The reflux device of claim 8, which includes a vent member connected to the first end of the reservoir tube.

11. The reflux device of claim 8, which includes a feeding tube connected to the delivery tube, wherein the feeding tube is received by the patient.

12. The reflux device of claim 8, wherein the measurement markings indicate a plurality of different volumes of reflux fluid.

13. A reflux device for use with an administration device during enteral feeding of a patient, the administration device having at least one administration tube, the reflux device comprising:
a reservoir tube having a first end defining at least one opening, a body having a predetermined diameter and a second end defining an opening;
a multi-way connector in fluid communication with the second end of the reservoir tube and the administration tube; and
a first delivery tube in fluid communication with the multi-way connector; and
a second delivery tube connected to the first delivery tube, the second delivery tube received directly or indirectly by the patient, the second delivery tube having a diameter which is less than the diameter of the body of the reservoir tube.

14. The reflux device of claim 13, wherein the second end of the reservoir tube includes an adapter member having an opening which is less than the diameter of the body of the reservoir tube.

15. The reflux device of claim 13, wherein the multi-way connector is removably connected to the administration tube.

16. The reflux device of claim 13, wherein the multi-way connector is rigidly connected to the administration tube.

17. The reflux device of claim 13, which includes a third delivery tube having a first end connected to the second end of the reservoir tube and a second end connected to the multi-way connector.

18. The reflux device of claim 13, wherein the second delivery tube includes a feeding tube.

19. The reflux device of claim 18, wherein the feeding tube includes a multi-way connector in fluid communication with the first delivery tube.

20. The reflux device of claim 13, wherein at least part of the multi-way connector is positioned at or below a patient level.

21. A method of enterally feeding a patient, the method comprising the steps of:
(a) delivering feeding fluid from a feeding source through a delivery tube to a patient;
(b) receiving reflux fluid from the patient through the delivery tube;
(c) channeling the received reflux fluid to a reservoir tube; and
(d) enabling any gaseous portion of the reflux fluid to vent to atmosphere.

22. The method of claim 21, which includes the step of using markings on the reservoir tube to gather information about reflux fluid.

23. The method of claim 22, which includes the step of maintaining a constant presence of fluid in the delivery tube.

24. The method of claim 21, which includes the step of returning reflux fluid to the patient when predetermined pressure conditions are present.

25. The method of claim 21, which includes the step of enabling a patient to expel all of the patient's reflux fluid into the reservoir tube during an entire enteral feeding process.

26. A method of treating reflux fluid during enteral feeding, said method comprising the steps of:
(a) maintaining a pressure in a reservoir tube which is less than a patient's expulsion pressure;
(b) enabling a patient to expel reflux fluid into the reservoir tube;
(c) receiving reflux fluid in the reservoir tube;
(d) enabling any gaseous portion of the received reflux fluid to flow out of the reservoir tube to atmosphere;
(e) using measurement markings on the reservoir tube to gather information about the reflux fluid in the reservoir tube; and
(f) enabling at least part of the reflux fluid in the reservoir tube to be delivered from the reservoir tube through a delivery tube to the patient after predetermined pressure conditions have been satisfied.

27. The method of claim 26, which includes the step of enabling the patient to expel various amounts of reflux fluid until enteral feeding is terminated.

28. The method of claim 27, which includes the step of receiving all of said various amounts of reflux fluid in the reservoir tube during an entire enteral feeding period.

29. An enteral feeding unit comprising:
a container which contains feeding fluid, the container defining at least one opening;
an administration tube having a first end and a second end, the first end of the administration tube connected directly or indirectly to the container and the second end of the administration tube in fluid communication with a reservoir tube, the reservoir tube having a first end defining at least one opening, a body having a predetermined diameter and a second end defining an opening; and
a delivery tube in fluid communication with the second end of the reservoir tube, the delivery tube received directly or indirectly by the patient and the delivery tube having a diameter which is less than the diameter of the body of the reservoir tube.

30. The enteral feeding unit of claim 29, wherein the reservoir tube is non-collapsible.

31. The enteral feeding unit of claim 29, wherein the reservoir tube has a volume which is large enough to receive all reflux fluid expelled by a patient during an entire enteral feeding process.

32. The enteral feeding unit of claim 29, which includes a plurality of measurement markings on the reservoir tube.

33. The enteral feeding unit of claim 29, which includes a vent member connected to the first end of the reservoir tube.

34. The enteral feeding unit of claim 29, which includes a feeding tube connected to the delivery tube, wherein the feeding tube is received by the patient.

35. The enteral feeding unit of claim 29, which includes a pump which co-acts with the administration tube.

36. The enteral feeding unit of claim 29, wherein the delivery tube has a first end connected to the reservoir tube and the administration tube and a second received directly or indirectly by the patient, said connection of the first end to the reservoir tube and the administration tube positioned at or below a patient level.

37. The enteral feeding unit of claim 29, wherein the delivery tube is positioned relative to the patient so that there is a constant presence of fluid in the delivery tube.
